(19) United States
(12) Patent Application Publication
(10) Pub. No.: US 2026/0165963 A1
(43) Pub. Date: Jun. 18, 2026

(54) SELF-ASSEMBLED BIODEGRADABLE STIMULI-RESPONSIVE MICROGELS

(71) Applicants: UNIVERSITE DE PAU ET DES PAYS DE L'ADOUR, Pau (FR); CENTRE NATIONAL DE LA RECHERCHE SCIENTIFIQUE, Paris (FR)

(72) Inventors: Laurent BILLON, Saint Faust (FR); Garbine AGUIRRE UGARTE, Lons (FR)

(21) Appl. No.: 19/125,097

(22) PCT Filed: Oct. 25, 2023
(86) PCT No.: PCT/EP2023/079827

§ 371 Date: Apr. 28, 2025

(30) Foreign Application Priority Data
Oct. 28, 2022 (EP) 22306638.2

(51) Int. Cl. A61K 9/06; A61K 8/02; A61K 8/04; A61K 8/73; A61K 8/86; A61K 9/16

(52) U.S. Cl. CPC A61K 9/06; A61K 8/0216; A61K 8/042; A61K 8/736; A61K 8/86; A61K 9/1641; A61K 9/1652

(57) ABSTRACT

The present invention relates to a biodegradable microgel composition comprising an oligo (ethylene glycol)-based copolymers crosslinked with a modified chitosan cross-linker obtainable by reaction of a chitosan with monomers comprising —COOH or —COO-M⁺ groups, M⁻ representing a cation, wherein said microgel composition comprises microgel particles. This microgel composition is able to form cohesive films spontaneously, which are biodegradable and feature mechano-electrical properties which are useful for the delivery of cosmetic or therapeutic agents of different nature.

### FIELD OF THE INVENTION

### Background of the Invention

The primary challenge of topical treatment is the penetration of compounds for cosmetic and therapeutic applications through the primary skin barrier known as stratum corneum. In the last years, several techniques and formulations have been developed with the aim of overcoming skin barriers by favoring the compound penetration into the deeper layers of the skin.

In this regard, among different approaches developed to increase skin permeability, iontophoresis is a relevant technique. This technique is based on the application of an electric field with a low electrical potential difference between the skin and the delivery systems to enhance the delivery across the skin through preexisting pores of the stratum corneum or through new pores. In this scenario, materials able to intrinsically generate an electric filed by soft and slight mechanical deformations are very interesting being able to break the skin barrier while delivering therapeutic molecules due to mechanical deformations. In the last years, several authors have attempted to accurately study the mechano-electrical properties of soft ionic macroscopic hydrogels.

Biocompatible and multi-responsive oligo (ethylene glycol)-based microgels have been synthesized. Said microgel can form Self-Assembled Microgel Films (SAMF) presenting mechano-electrical behavior, wherein an output voltage can be observed by compressing those films (WO2016/110615). Moreover, it has been demonstrated the opportunity to encapsulate active molecules together with inorganic nanoparticles (Boularas et al. 2015, Macromol. Rapid Commun., 36:79) by such colloidal system enhancing their potential applications as delivery systems. The effective encapsulation of active molecules has been also observed in the case of Self-Assembled Microgel Films (SAMF).

However, obtaining biodegradable delivery systems remains a key objective both for therapeutic and cosmetic applications. Additionally, tuning and adapting the properties of said microgels and films to deliver cosmetic and therapeutic agents depending on the desired application is also a key objective, which could allow, for instance, a controlled release of said therapeutic and cosmetic agents during time through preexisting or new pores of the stratum corneum, in order to provide a sustained cosmetic or therapeutic effect, or tuning the mechanical or adhesive behaviors of the formed films, among others.

### SUMMARY OF THE INVENTION

The inventors have synthetized stimuli-responsive microgels comprising particles with a core-shell structure, which are biodegradable and are able to spontaneously self-assemble forming cohesive films. Said microgels, and the films formed with said microgels of the invention, are able to entrap and release different type of active molecules, including macromolecular compounds. The mechano-electrical properties of the microgels and films formed therefrom, make them useful in the delivery of active substances through preexisting or new pores of the stratum corneum.

Thus, the present invention relates to a microgel composition comprising microgel particles, wherein said microgel particles comprise oligo (ethylene glycol)-based copolymers crosslinked with a modified chitosan cross-linker obtainable by reaction of a chitosan with monomers comprising —COOH or —COO⁻M⁺ groups, M⁺ representing a cation. The modified chitosan cross-linker comprises, accordingly, monomers covalently linked to the chitosan chain by ester groups or amide groups resulting from reacting the COOH or COO⁻M⁺ groups of the monomers with, respectively, hydroxyl or primary amine groups of the chitosan chain.

The modified chitosan cross-linker obtainable by reaction of a chitosan with monomers comprising —COOH or —COO⁻M⁺ groups, M⁺ representing a cation, allows modulating the properties of the microgels, either modifying the number of covalent reticulation points (number of COOH or COO⁻ M groups covalently attached to the chitosan chains), or by modifying the percentage or ratio of the modified chitosan cross-linker in relation to the monomers of the microgel.

The invention also concerns a process of preparing a microgel composition according to the present invention, said process comprising preparing a microgel via precipitation polymerization of monomers selected among:
di(ethylene glycol) methyl ether methacrylate (MeO2MA);
an oligo (ethylene glycol) methyl ether methacrylate (M(EO)nMA), n being an integer ranging from 3 to 12, preferably ranging from 8 to 10;
   wherein said monomers are crosslinked with a modified chitosan cross-linker obtainable by reacting a chitosan with one or more monomers of formula CR₁R₂═CR₃R₄ in which R₁, R₂, R₃ and R₄ represent a hydrogen, a halogen or a hydrocarbon group, at least one of the four groups comprising a —COOH or —COO′M⁺ group, M⁺ representing a cation; and wherein said microgel comprises particles.

The microgels according to the invention are capable of self-assembling in order to form a film consisting of one or more layers of microgels, by a process of drying or evaporating an aqueous suspension of said microgels.

Hence, in another aspect, the present invention relates to a Self-Assembled Microgel Film obtained by solvent evaporation, and to a Self-Assembled Microgel Film for use in a method, for example in a cosmetic method or in a therapeutic method, comprising a step of applying on keratin materials a microgel composition according to the invention.

Advantageously, the films formed according to the present invention generate an electric potential via compression effect with an applied compression force of about 10 to 15 N, which is a range equivalent to that of one that could be applied by a finger for the application of a product onto the skin (for example, cream application). Output voltages higher than 250 mV were generated by compression and maintained constant almost for 1 minute.

Additionally, the films formed according to the present invention may be connected in series allowing the generation of higher electrical potentials by compression, being possible to amplify and linearly tune it combining the appropriate number of films in series. Accordingly, another aspect of the present invention refers to a series of films obtained by drying or evaporating solvent of a microgel composition according to the present invention, or to a series of Self-Assembled Microgel Films according to the present invention, wherein each film or each Self-Assembled Microgel Film, is connected respectively to another film or Self-Assembled Microgel Film.

From the point of view of cosmetic and therapeutic applications, reversible pore induction in cell membranes and lipid bilayer membranes has been observed at 150-250 mV (several seconds) (H. Inada, A.-H. Ghanem, W. I. Higure, Pharm. Res., 1994, 11, 687-697). Therefore, the films formed according to the present invention should be able to create new pores in the skin enhancing the penetration of the active molecules.

Hence, the present invention also relates to a cosmetic product comprising a microgel composition according to the present invention and at least a cosmetic agent, wherein the particles of the microgel comprise the cosmetic agent and, as well, the present invention relates to a make-up or a skin care method comprising a step of applying on keratinous materials such a cosmetic product.

In a last aspect, the present invention concerns a therapeutic product comprising a microgel composition according to the present invention and a therapeutic agent, wherein the particles of the microgel comprise the therapeutic agent, the use of said therapeutic product in therapy as well as its use for delivering therapeutic agents through the stratum corneum.

### FIGURES

FIG. 1. ¹H NMR spectrum of a modified chitosan-methacrylate (Chi-MA) cross-linker.

FIG. 2. Average hydrodynamic diameters as a function of temperature and using Chi-MA20 as cross-linker (chitosan-methacrylate cross-linker having 20 methacrylate groups per chitosan polymer chain). ▪Heating and □cooling cycles. (2A) 1 mol %, (2B) 0.4 mol %, (2C) 0.2 mol %.

FIG. 3. Average hydrodynamic diameters as a function of temperature and using Chi-MA9 as cross-linker (modified chitosan-methacrylate cross-linker having 9 methacrylate groups per chitosan polymer chain). ▪Heating and □cooling cycles. (3A) 1 mol %, (3B) 0.4 mol %, (3C) 0.2 mol %.

FIG. 4. Average hydrodynamic diameters as a function of temperature for different microgels. (4A) GA-Chi9/2.2; (4B) GA-Chi2/10.

FIG. 5. Atomic Force Miscroscopy (AFM) micrographs of GA-Chi2/10 microgel particles at room temperature (5A) and 37° C. (5B).

FIG. 6. Swelling ratio of microgel particles (6A) and hydrodynamic diameter of oligosaccharides (6B) after enzymatic degradation.

FIG. 7. AFM images in dried state of GA-Chi20/1 (7A), GA-Chi9/2.2 (7B) and GA-Chi2/10 (7C) microgels before and after enzymatic degradation.

FIG. 8. Swelling ratio of films prepared with microgels GA-Chi20/1, GA-Chi9/2 and GA-Chi2/10 (8A) and hydrodynamic diameter of oligosaccharides (8B) after enzymatic degradation.

FIG. 9. Output voltage (mV) generated after oscillatory finger compression for one film (9A) and 2 films in series (9B). GA-Chi20/1 (continuous line). GA-Chi9/2.2 (dashed line).

FIG. 10. Stress-strain curves of films formed with microgels of the invention GA-Chi20/1, GA-Chi9/2,2 and GA-Chi2/10 performed at a crosshead speed of 1 mm·s⁻¹.

FIG. 11. Stress-strain curves of adhesive films formed with microgels of the invention GA-Chi20/1 and GA-Chi9/2.

### DETAILED DESCRIPTION OF THE INVENTION

In a first aspect, the present invention concerns a microgel composition comprising microgel particles, wherein said microgel particles comprise oligo (ethylene glycol)-based copolymers crosslinked with a modified chitosan cross-linker obtainable by reaction of a chitosan with monomers comprising —COOH or —COO⁻M⁺ groups, M⁺ representing a cation.

### Microgel

“Microgel” in the sense of the invention are compositions (microgel compositions) in the form of an aqueous dispersion of microgel particles or in the form of a film comprising microgel particles, wherein the microgel particles are crosslinked polymer in the form of particles having a size that varies from 100 nm to 500 nm in the dry state (i.e. containing less than 2% by weight of water), preferably between 125 and 450 nm, preferably between 150 and 250 nm, more preferably of the order of 200 nm. Typically, the particles are spherical.

It must be noted that microgel is distinct from a hydrogel. Hydrogel is a bulky material chemically formed without any possibilities of re-shaping it. Microgel is particles in colloidal state dispersed in water media. Such colloidal solution can be in-situ shaped by drying the solution without any chemical reaction but only physical-chemical interactions.

In an embodiment, the microgel particles comprise oligo (ethylene glycol)-based copolymers crosslinked with a modified chitosan cross-linker obtainable by reaction of a chitosan with monomers comprising —COOH or —COO⁻M⁺ groups, M⁺ representing a cation. The cation M⁺ may be any suitable for the purposes of the present invention, for example, Na⁺, K⁺, NH₄⁺, among others.

In an embodiment the modified chitosan cross-linker comprises substitutions selected from the group consisting of acrylamide, methacrylamide, acrylate, and methacrylate groups, or a mixture thereof.

In an embodiment, the oligo (ethylene glycol)-based copolymers comprise di(ethylene glycol) methyl ether groups and oligo (ethylene glycol) methyl ether groups having from 3 to 12 ethylene glycol units.

In that connection, the microgels of the invention are obtainable by aqueous phase precipitation polymerization of one or more of the following monomers:
di(ethylene glycol) methyl ether methacrylate (MeO₂MA); and
an oligo (ethylene glycol) methyl ether methacrylate (M(EO)ₙMA), n being an integer ranging from 3 to 12, preferably ranging from 8 to 10;
   cross-linked with a modified chitosan cross-linker obtainable by reacting a chitosan with one or more monomers of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation.

MeO₂MA represents for example 50 mol % to 90 mol % of the total number of moles of the monomers, M(EO)ₙMA preferably represents 10 to 50 mol % of the total number of moles of the monomers and the modified chitosan cross-linker preferably represents 0.1 mol % to 20 mol % of the total number of moles of the monomers, the sum of these three contents being equal to 100%.

The molar ratio between MeO₂MA and M(EO)ₙMA is preferably between 1:1 and 20:1, for example between 5:1 and 10:1.

According to one embodiment, MeO₂MA represents for example 80 to 90 mol % of the total number of moles of the three monomers, M(EO)ₙMA preferably represents 5 to 15 mol % of the total number of moles of the monomers and the modified chitosan cross-linker preferably represents 0.1 to 10 mol % of the total number of moles of the monomers, the sum of these three contents being equal to 100%.

In an embodiment, M(EO)ₙMA is preferably an oligo (ethylene glycol) methyl ether methacrylate also denoted OEGMA.

Acrylic acid may be excluded from the definition of the monomer of formula CR₁R₂═CR₃R₄ in certain cases.

In a particular embodiment, microgels are obtainable aqueous phase precipitation polymerization of monomers cross-linked with a modified chitosan cross-linker, said monomers being di(ethylene glycol) methyl ether methacrylate (MeO₂MA), oligo (ethylene glycol) methyl ether methacrylate (OEGMA); and wherein the modified chitosan cross-linker is obtained by reacting a chitosan with methacrylic acid (MAA) monomers.

In a yet particular embodiment, MeO₂MA represents for example 80 to 90 mol % of the total number of moles of the three monomers, OEGMA represents 5 to 15 mol % of the total number of moles of the monomers and the modified chitosan cross-linker preferably represents 0.1 to 10 mol % of the total number of moles of the monomers, the sum of these three contents being equal to 100%.

### Modified Chitosan Cross-Linker

According to the present invention, the modified chitosan cross-linker obtainable by reaction of a chitosan with monomers comprising —COOH or —COO⁻M⁺ groups, M⁺ representing a cation, is the sole cross-linker.

Hence and as used herein, the term “modified chitosan cross-linker” refers to a chitosan-based polymer which has been covalently modified by reacting a chitosan polymer with monomers comprising —COOH or —COO⁻M⁺ groups, M⁺ representing a cation, for example by reacting a chitosan polymer with one or more monomers of formula CR₁R₂═CR₃R₄ (vinylic monomers) in which R₁, R₂, R₃ and R₄ represent a hydrogen, a halogen or a hydrocarbon group, at least one of the four groups comprising a —COOH or —COO⁻M⁺ group, M⁺ representing a cation, as described in the present invention. In a particular embodiment said modified chitosan cross-linker is obtained by reacting a chitosan with methacrylic acid (MAA) monomers.

As used herein “chitosan” or “chitosan polymer” refers to any linear polysaccharide made up of arbitrarily distributed β-(1-4)-linked D-glucosamine (deacetylated) and N-acetyl-D-glucosamine (acetylated).

The term “deacetylation degree” refers herein to the molar fraction of D-glucosamine present in the chitosan polymer. Examples of chitosan used to obtain the modified chitosan cross-linkers of the microgels of the present invention are chitosan polymers featuring a deacetylation degree of at least 50%, preferably of at least 80% and more preferably of 90%; and a molecular weight ranging from 10-200 kDa, preferably a molecular weight lower than 100 kDa, more preferably a molecular weight lower than 50 kDa and even more preferably a molecular weight of 10-20 kDa. In a particularly preferred embodiment the chitosan polymer features a deacetylation degree of 90% and a molecular weight of 10-20 kDa.

The monomer of formula CR₁R₂═CR₃R₄ used to obtain the modified chitosan cross-linker is preferably such that R₁ and R₂ each represent a hydrogen, R₃ represents H or an alkyl group, preferably a C₁-C₆ alkyl group, optionally substituted with —OH or —COOH, and R₄ represents, independently of R₃, the —COOH group or an alkyl group, preferably a C₁-C₆ alkyl group, optionally substituted with —OH or —COOH. The alkyl group may be methyl, ethyl or n-butyl. According to one particular embodiment, R₁ and R₂ each represent a hydrogen and R₃ and R₄ independently represent —H, —COOH, or COOH.

Said monomer of formula CR₁R₂═CR₃R₄ may for example be chosen from methyl acrylic, methyl methacrylic, ethyl acrylic, ethyl methacrylic, n-butyl acrylic and n-butyl methacrylic acids, vinylic monomer comprising a carboxylic group.

According to one embodiment, the monomer of formula CR₁R₂═CR₃R₄ may be methacrylic acid or itaconic acid.

As used herein, the “degree of substitution” or DS refers to the number-COOH or —COO⁻M⁺ groups covalently linked to each chitosan chain forming ester groups or amide groups resulting from the reaction of the COOH or COO⁻M groups with, respectively, hydroxyl or primary amine groups of each chitosan chain present in the modified chitosan cross-linker; for example DS refers to the number of vinylic groups comprising —COOH or —COO⁻M⁺ groups covalently linked to each chitosan chain, and in particular the number of acrylate, acrylamide, methacrylate and methacrylamide groups, or a mixture thereof, covalently linked to each chitosan chain present in the modified chitosan cross-linker.

In an embodiment, the degree of substitution of the modified chitosan cross-linker is at least 2, preferably from 2 to 20.

Advantageously, using the modified chitosan cross-linker as defined herein, microgels could be obtained by using aqueous phase precipitation polymerization without addition of any other cross-linker and surfactant stabilizer.

In an embodiment, the modified chitosan cross-linker comprises substitutions selected from the group consisting of an acrylamide, methacrylamide, acrylate, and methacrylate groups, or a mixture thereof, preferably, said modified chitosan cross-linker is a covalently modified chitosan comprising at least 2, preferably 2 to 30, even more preferably 2 to 20 substitutions per each chitosan polymer chain, being said substitutions selected from the group consisting of acrylamide, methacrylamide, acrylate and methacrylate groups, or a mixture thereof.

In an embodiment, the modified chitosan cross-linker represents from 0.1 to 20 mol % with respect to the total number of moles of di(ethylene glycol) methyl ether methacrylate (MeO₂MA), oligo (ethylene glycol) methyl ether methacrylate (OEGMA) and modified chitosan cross-linker. The monomers are those previously described herein before.

The microgel particles feature a core/shell structure, wherein the inner structure of the microgels particles can depend on the degree of substitution of the modified chitosan crosslinker used. In fact, as illustrated in example 2, the number of subchains (polymeric chains between two cross-linking points) in the shell is lower that the number of subchains found in the core, except for microgels obtained with modified chitosan cross-linkers with a low degree of substitution, in which the number of subchains found in the shell is higher than in the core. This means that the cross-linking points distribution in the core and shell portions of the microgel particles changes in function of the degree of substitution (DS) of the modified chitosan cross-linker.

Accordingly, an embodiment refers to a microgel composition according to the invention comprising microgel particles comprising oligo (ethylene glycol)-based copolymers crosslinked with a modified chitosan cross-linker obtainable by reaction of a chitosan with monomers comprising —COOH or —COO⁻M groups, M⁺ representing a cation, wherein the modified chitosan cross-linker comprises a degree of substitution higher than 5, more preferably between 8 and 30, and more preferably between 9 and 20, wherein said particles have a core/shell structure, and wherein the core comprises oligo (ethylene glycol)-based copolymers having a higher degree of crosslinking than the oligo (ethylene glycol)-based copolymers of the shell.

On the other hand, another embodiment refers to a microgel composition according to the invention comprising microgel particles comprising oligo (ethylene glycol)-based copolymers crosslinked with a modified chitosan cross-linker obtainable by reaction of a chitosan with monomers comprising —COOH or —COO⁻M⁺ groups, M⁺ representing a cation, wherein the modified chitosan cross-linker comprises a degree of substitution lower than 5, more preferably lower than 3, even more preferably having a degree of substitution of 2, wherein said particles have a core/shell structure, and wherein the shell comprises an oligo (ethylene glycol)-based copolymer having a higher degree of crosslinking than the oligo (ethylene glycol)-based copolymers of the core.

Additionally, the properties of the microgels of the invention may also be tuned in function of the mol % of modified chitosan cross-linker used to prepare the microgels according to the present invention.

The differences in the core/shell particle microstructure, resulting from the use of modified chitosan cross-linkers, as defined herein, with different degree of substitution, and in different concentrations, may be used for tuning the properties of the microgels of the present invention, in particular, the mechanical properties of the films formed with said microgels, their adhesive properties, or the encapsulation efficiency and release profile of different active molecules (small and macromolecules), as well as the thermal and pH dependency thereof, as shown in the examples.

### Biodegradable

The microgels of the invention are biodegradable. As used herein, the term “biodegradable” refers to the fact that the microgels of the invention may be degraded or broken down through natural processes, such as enzymatic processes. For instance, the microgels of the invention, and the films formed with them, may be degraded by enzymes, such as lysozyme, as shown in the examples. Lysozyme is a hydrolytic glycosidase [(β-) glycoside hydrolase] abundant in human secretions (tears, saliva, human milk, mucus) as well as in human macrophages and polymorphonuclear neutrophils (PMNs), which forms part of the innate immune system due to its capacity to lyse bacterial cell membranes.

### Process

In another aspect, the present invention relates to process of preparing a microgel composition according to the present invention, said process comprising preparing a microgel via precipitation polymerization of monomers selected among:
di(ethylene glycol) methyl ether methacrylate (MeO₂MA);
an oligo (ethylene glycol) methyl ether methacrylate (M(EO)ₙMA), n being an integer ranging from 3 to 12, preferably ranging from 8 to 10;
   wherein said monomers are crosslinked with a modified chitosan cross-linker obtainable by reacting a chitosan with one or more monomers of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation; and wherein said microgel comprises particles.

The monomers and the modified chitosan cross-linker are those previously described herein before.

In an embodiment, the modified chitosan cross-linker is obtained by reacting a chitosan with one or more monomers of formula CR₁R₂═CR₃R₄, as defined above herein, at pH between 5-6, preferably at pH 5.5, in the presence of N-ethyl-N′-(3-(dimethylamino) propyl) carbodiimide (EDC), N-hydroxysuccinimide (NHS), and a buffer, preferably a 2-(N-morpholino) ethanesulfonic acid buffer.

In an embodiment, the precipitation polymerization comprises a step of bringing into contact in an aqueous phase, in the presence of the modified chitosan cross-linker as defined herein, the monomers described above, at a temperature comprises between 40° C. and 90° C., and preferably at a temperature of 70° C.

In an embodiment, in the step of preparing microgel, the polymerization of the monomers may be initiated by addition of a water-soluble radical initiator, for example potassium persulfate (KPS) at a temperature comprises between 40° C. and 90° C., and preferably at a temperature of 70° C.

### Films, Self-Assembled Microgel Films, Process of Obtaining Films and Self-Assembled Microgel Films and Series of Self-Assembled Microgel Films

The microgels according to the invention are capable of assembling in order to form a film consisting of one or more layers of microgels, by a process of drying or evaporating an aqueous suspension of said microgels.

Another aspect of present invention refers, therefore, to a Self-Assembled Microgel Film, wherein said Self-Assembled Microgel Films is obtained by solvent evaporation of a microgel composition according to the present invention.

Another embodiment refers to a process of obtaining a Self-Assembled Microgel Film comprising a step of applying on keratin materials a microgel composition according to the present invention.

The thickness of each layer ranges from 10 to 1000 microns, preferably from 100 to 800 microns, more preferably from 100 to 400 microns or from 400 to 800 microns.

In an embodiment, the thickness of the layer is from 150-200 microns.

In another embodiment, the thickness of the layer is around 200 microns.

In an embodiment, the thickness of the film can be increased through the deposition of different layers onto keratin materials, preferably the skin.

Hence, the film can have a thickness that varies, ranging in some embodiments from 10 microns to 5.0 millimeters, preferably from 350 microns to 4.0 millimeters, preferably from 700 microns to 3.0 millimeters.

In an embodiment, the film area is comprised between 1E-05 m² and 2.5E-04 m². In an embodiment, the films are prepared by a process of drying or evaporating solvent at a temperature comprised between 20° C. and 40° C., preferably at 35° C.

In an embodiment, the films of microgel particles can be formed according to a step of placing an aqueous microgel dispersion prepared, for example according to the process described above into a mold, and a step of drying the water dispersion. Drying can be performed by placing the mold at a temperature higher than 30° C., preferably around 35° C. or being ambient temperature (i.e. between 15° C. and 30° C., for example between 18° C. and 25° C.).

Microgels compositions according to the invention are also capable of forming a cohesive and elastic films. It is not necessary in the context of the invention to encapsulate or support the microgels in order to form a film; consequently; interaction between the microgels and keratin materials on which they are formed after water evaporation of an aqueous dispersion of the microgel particles is optimal.

Hence, the invention also concerns a Self-Assembled Microgel Films for use in a method comprising a step of applying on keratin materials a composition according to the invention, wherein the said Self-Assembled Microgel Films is obtained by solvent evaporation of the composition.

In an embodiment, the self-assembled microgel film is obtained by simple drying at ambient temperature.

Keratin materials are selected among the skin, the scalp, the hair, the nail, the lips, the eyebrow or the mucosa. Preferably the composition according to the invention is applied onto the skin.

These microgel compositions of the invention may thus be used as film-forming agent in therapeutic or cosmetic compositions, so as to improve the hold of these compositions on keratin materials and enhance the penetration of the active agent through stratum corneum due to their mechanoelectrical properties.

The films formed by the microgels of the present invention may also feature adhesive properties. Thus, an embodiment refers to a film having adhesive properties, wherein said film is formed by solvent evaporation of a microgel composition according to the invention, wherein said microgel composition comprises microgel particles comprising oligo (ethylene glycol)-based copolymers crosslinked with a modified chitosan cross-linker as defined herein, obtainable by reaction of a chitosan with monomers comprising —COOH or —COO⁻M⁺ groups, M⁺ representing a cation, wherein the modified chitosan cross-linker comprises a degree of substitution higher than 5, more preferably between 8 and 30, and more preferably between 9 and 20, wherein said particles have a core/shell structure, and wherein the core comprises oligo (ethylene glycol)-based copolymers having a higher degree of crosslinking than the oligo (ethylene glycol)-based copolymers of the shell.

### Series of Self-Assembled Microgel Films

In an embodiment, and with the aim of increasing the electrical potential generated, multiple films or Self-Assembled Microgel Films can be connected in series.

Hence, in an embodiment, the present invention also relates to a series of films or Self-Assembled Microgel Films wherein each film or Self-Assembled Microgel Film is connected respectively to another film or Self-Assembled Microgel Film.

In an embodiment, films or Self-Assembled Microgel Films are connected through an electrical cable or wire linking the bottom electrode of one film or Self-Assembled Microgel Film with the upper electrode of another one.

In an embodiment, 2 to 50 films or Self-Assembled Microgel Films are connected, preferably 2 to 10, more preferably, 2 to 6 films or Self-Assembled Microgel Films are connected.

Advantageously, when using a series of films or Self-Assembled Microgel Films, a high electrical potential can be generated, being therefore possible to tune and amplify said potential combining the appropriate number of films in series.

### Mechanoelectrical Properties

The composition and the films formed according to the present invention generate an electric potential via compression effect.

Output voltages generated after finger compression for microgel self-assembled films according to the invention were around 270 mV and even superior to 400 mV in some cases. The output voltage is also maintained constant for a period of about 1 minute.

From the point of view of cosmetic and therapeutic applications, reversible pore induction in cell membranes and lipid bilayer membranes has been observed at 150-250 mV (several seconds) (H. Inada, A.-H. Ghanem, W. I. Higure, Pharm. Res., 1994, 11, 687-697). Therefore, the films formed according the present invention should be able to create new pores in the skin enhancing the penetration of the active molecules.

Several layers of the film can be deposed onto keratin materials, preferably the skin, to increase the output voltage generated.

These properties make possible to envisage the use of the microgel compositions of the invention, as well as the use of the films formed by solvent evaporation of said microgel compositions, for the preparation of a cosmetic or a pharmaceutical product. These microgel compositions of the invention, as well as the films formed by solvent evaporation of said microgel compositions, are able to stimulate keratin materials, preferably the skin, in order to deliver cosmetic or therapeutic agents entrapped or loaded therein, via compression effect.

### Active Agent Loaded Microgel

In an embodiment, the microgels are loaded with an active agent. By “loaded” is meant that the microgel particles include an amount of an active agent(s). As such, an amount of active agent is present in the microgel particle and may be viewed as entrapped in the microgel particle. The term “entrapped” means that the active agent is located within the polymer network of the microgel. The network of the crosslinked polymer can form a barrier around the active-agent that can be suppressed by some physical change in the network. The entrapped active agent may not be linked to the crosslinked polymer with a covalent bond. The entrapped active agent can have electrostatic interactions, Van der Walls bonds or hydrogen bonds with the crosslinked polymer, that can be engaged between C═C bonds of —OH groups of the organic molecules and ethylene glycol moieties of the crosslinked polymer.

Microgels according to the invention can advantageously entrap active agent and encapsulate high amounts of different molecules.

In fact, as seen in the examples, the microgel particles of the invention are swollen when the temperature of the medium in which said particles are placed decreases, whereas at temperatures above the VPTT the particles collapse. The release of the active molecules will depend on the type of interactions between said active molecules and the microgel particles of the invention: if the interactions are hydrophobic the release will occur when the microgel particles are swollen, whereas if the interactions are electrostatic the release will happen when the microgel particles are collapsed.

Moreover, as shown in example 3, the microgel particles of the invention feature a non-sharp transition from swollen to collapsed state. Accordingly, microgel particles featuring at least 0.2 mol % of a modified chitosan cross-linker, as defined herein obtainable by reaction of a chitosan with monomers comprising —COOH or —COO⁻M⁺ groups, M⁺ representing a cation, with a degree of substitution of at least 2, show non-conventional thermal behavior, i.e., the microgel particles collapse progressively within a range of temperatures, which may allow the use of the microgels of the invention as slow-release delivery systems.

In an embodiment, active agent are hydrophobic molecules.

In an embodiment, the active agent can be a cosmetic agent or a therapeutic agent.

The “amount of the active agent in the loaded microgel” is the weight (in microgram (μg)) of the active agent that is entrapped in the crosslinked polymer per 1 mg of crosslinked polymer in the loaded microgel. The “amount of the active agent in the loaded microgel” is also mentioned as the “entrapped substance amount” in the rest of the description.

Preparation of active-agent loaded microgel has been described in patent application WO2019/07740. Briefly, active-agent loaded microgel can be prepared according to the steps of:
preparing a dispersion of unloaded microgel particles in water
preparing a feeding solution of the active agent;
mixing the microgel obtained and the solution of the active agent causing encapsulation of the active agent in the microgel particles; and
recovering active-agent loaded microgel particles.

Advantageously, unloaded microgel particles are prepared by a precipitation polymerization method as described in the present invention.

Mixing step of active substance solution and unloaded microgel dispersion preferably comprises a step of heating at a temperature that is higher than the volume phase transition temperature (VPTT) of the unloaded microgel particles, and a step of cooling the obtained dispersion of loaded microgels at ambient temperature (25° C.).

The feeding solution of the active agent can be obtained by dissolution of a determined amount of the active agent in an appropriate solvent. Complete dissolution of a determined amount of the active substance in the solvent can be performed at a temperature being from ambient temperature to a temperature that is above the volume phase transition temperature of the unloaded microgel particles.

The “amount of the active agent in the feeding solution” also called “the feeding substance amount” in the following description is the weight of the active agent in the feeding solution (in μg or mg) per 1 mg of unloaded microgel particles that are used to entrap the active substance. The feeding substance amount unit may be written in a shorter way “mg/mg” or “microgram/mg”.

As described in WO 2019/077404, this process enables a high Entrapment Efficiency EE %.

The Entrapment Efficiency (EE %) is defined as the ratio of the weight of the active agent that is entrapped in the loaded microgels and the amount of the active agent that is contained in the feeding solution. The Entrapment Efficiency (EE %) can also be defined as the ratio A/B of the entrapped substance amount (A) and the feeding substance amount (B), as defined in the present application.

Active agent can be encapsulated into microgels that are in the form of an aqueous dispersion, or into microgels that have been prepared in the form of a film according to the description above.

Typically, the process for the preparation of active agent loaded microgel in the form of a film comprises the step of:
preparing a feeding solution of the active agent in a solvent,
preparing a film of unloaded microgel particles,
immersing the film in the feeding solution so as to cause swelling of the film and diffusion of the active substance into the film, and
recovering the microgels that can be in the form of an active substance loaded microgel film.

The films are prepared according to the present invention.

The step of immersing the film can be performed at 25° C. for at least 12 hours or 24 hours.

### Cosmetic Product

In an embodiment, the present invention concerns a cosmetic product comprising a microgel composition as described above and at least one cosmetic agent.

In an embodiment, the microgel particles entrap the cosmetic agent. Microgels then can be named “loaded microgels” or “loaded microgel particles”.

The cosmetic agent includes but is not limited to chemicals, compounds, small or large molecules (macromolecules), extracts, formulations or combinations that are known to induce or cause at least one effect on keratinous materials and/or in a skin tissue.

The microgel of the composition according to the invention can be in the form of an aqueous dispersion or in the form of film or a series of films.

The cosmetic composition can be in the form of a make-up product, a skin care product, a hair care product.

As long as the purpose and effect of the present invention are not impaired, cosmetic product of the present invention can further contain any acceptable excipients, in addition to the composition of the present invention.

### Make-Up or Skin Care Method

In an embodiment, the present invention also relates to a cosmetic (non-therapeutic) make-up or a skin care method comprising a step of applying on keratinous materials, a cosmetic product as described above, and applying a compression on said product.

In an embodiment, the make-up or skin care method comprises the following steps:
applying on keratinous materials a cosmetic product as described above, the microgel of the composition being in the form of an aqueous dispersion or a series of aqueous dispersion;
waiting in order to obtained by solvent evaporation a self-assembled microgel film;
optionally connecting a series self-assembled microgel films
applying a compression on the self-assembled microgel film obtained.

In an embodiment, the make-up or skin care method comprises the following steps:
applying on keratinous materials a cosmetic product as described above, the microgel of the composition being in the form of a film or a series of films;
optionally connecting the series of films
applying a compression on the film.

Output voltages generated after compression of the product comprising the conductive microgel according to the invention stimulate keratin materials and allow to deliver cosmetic material through stratum corneum into superficial layers of the skin.

Typically, the applied force or applied compression is around 10 to 15 N being this value range similar to that applied with a finger during the common application of a cream.

All the features applying to the composition according to the invention and all the features applying to the cosmetic product that have been described before also apply to the make-up or skin care method.

### Therapeutic Product

In an embodiment, the present invention also related to a therapeutic product comprising a microgel composition according to the invention and at least one therapeutic agent.

The microgel of the composition according to the invention can be in the form of an aqueous dispersion or in the form of film or a series of films.

The phrase “therapeutic agent” “which is interchangeably referred to herein as “drug” or “active agent” or therapeutically active agent”, describes a compound which exhibits a beneficial pharmacological effect when administered to a subject and hence can be used as a medicament, i.e., can be used in the treatment of a disease or condition that benefits from this pharmacological effect. As long as the purpose and effect of the present invention are not impaired, therapeutic product according the present invention contains at least one therapeutic agent and optionally any acceptable excipients, in addition to the composition of the present invention.

In an embodiment, the microgel particles entrap the therapeutic agent. Microgels then can be named “loaded microgels” or “loaded microgel particles”.

Output voltages generated after compression of the product comprising the conductive microgel according to the invention stimulate keratin materials and allow to deliver therapeutic agent through stratum corneum into superficial and deep layers of the skin in order to deliver the therapeutic agent.

### A Method for Delivering an Active Agent

In an embodiment, the present invention also concerns a method for delivering a therapeutic agent comprising a step of applying a therapeutic product comprising a microgel composition according to the invention and at least one therapeutic agent, as described above, on keratin materials of a subject and applying a compression on said product. In an embodiment the present invention refers to a self-assembled microgel film for use in a method for delivering a therapeutic agent comprising a step of applying a therapeutic product comprising a microgel composition according to the invention and at least one therapeutic agent, as described above, on keratin materials of a subject and applying a compression on said product.

In yet an embodiment, the present invention also concerns a method for delivering a cosmetic agent comprising a step of applying a cosmetic product comprising a microgel composition according to the invention and at least one cosmetic agent, as described above, on keratin materials and applying a compression on said product.

In an embodiment, the method for delivering or administering a cosmetic agent or a therapeutic agent to a subject comprises the following steps:
applying a cosmetic agent or a therapeutic product as described above on keratinous materials of a subject, the microgel of the composition being in the form of an aqueous dispersion or series of aqueous dispersion;
waiting in order to obtained by solvent evaporation a self-assembled microgel film;
eventually connecting self-assembled microgel films;
applying a compression on the self-assembled microgel film obtained.

In another embodiment, the method for delivering or administering a cosmetic agent or a therapeutic agent comprises the following steps:
applying a cosmetic agent or a therapeutic product as described above on keratinous materials of a subject, the microgel of the composition being in the form of a film or a series of film;
eventually connecting the series of film;
applying a compression on the film.

In yet an embodiment, the present invention also related to a therapeutic agent for use in therapy, wherein said therapeutic agent is delivered to a subject via the microgel composition according to the invention. In other words, an embodiment of the present invention refers to a therapeutic agent for use in therapy characterized in that said therapeutic agent is administered to a subject in a microgel composition according to the present invention.

In other words, it is also disclosed a method of treating or preventing a disease or condition in a subject in need thereof, wherein said method comprises administering to said subject an effective amount of a therapeutic agent for treating said disease or condition, wherein said therapeutic agent is administered to said subject via the microgel composition according to the invention. Also discloses is the use of a therapeutic agent for the manufacturing of a medicament, wherein said medicament comprises a microgel composition according to the invention and said therapeutic agent.

Said subject may be a human or an animal. An effective amount, according to the present disclosure is an amount that produces a beneficial pharmacological effect when administered to a subject. Said effective amount may vary depending on the therapeutic agent, the disease and its severity, and the age, weight, etc., of the subject to be treated.

In an embodiment, the therapeutic agent for use in therapy is administered to a subject via the following steps:
applying a therapeutic product as described above on keratinous materials of a subject, the microgel of the composition being in the form of an aqueous dispersion or series of aqueous dispersion;
waiting in order to obtained by solvent evaporation a self-assembled microgel film;
eventually connecting self-assembled microgel films
applying a compression on the self-assembled microgel film obtained.

In yet another embodiment, the therapeutic agent for use in therapy is administered to a subject via the following steps:
applying a therapeutic product as described above on keratinous materials of a subject, the microgel of the composition being in the form of a film or a series of film;
eventually connecting the series of film
applying a compression on the film.

Output voltages generated after compression of the product comprising the conductive microgel according to the invention stimulate keratin materials and allow to deliver therapeutic material through stratum corneum into superficial and deep layers of the skin.

Typically, the applied force or applied compression is around 10 to 15 N.

All the features applying to the composition according to the invention and all the features applying to the therapeutic product that have been described before also apply to the method for delivering the therapeutic agent.

### EXAMPLES

In the following examples, the following reagents were used.

TABLE 1
Chemical structures, names and abbreviations of reagents used.

| Chemical structure | Name | Abbreviation |
|---|---|---|
| 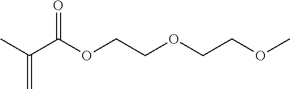 | Di(ethylene glycol) methyl ether methacrylate | MeO₂MA |
| 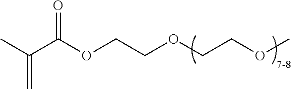 | Oligo(ethylene glycol) methyl ether methacrylate | OEGMA |
| 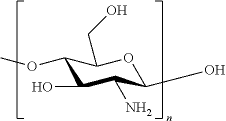 | Chitosan | Chi |
| 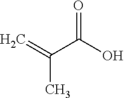 | Methacrylic acid | MAA |
| 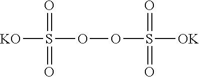 | Potassium persulfate | KPS |
| 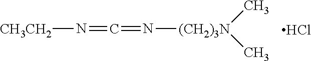 | N-(3-(dimethylamino)propyl)-N′- ethylcarbodiimide hydrochloride | EDC |
| 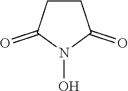 | N-hydroxy succinimmide | NHS |
| 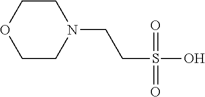 [SMILES: O=S(=O)(O)CCN1CCOCC1] | 2-(N-Morpholino) ethanesulfonic acid | MES |

### Example 1. Synthesis and Characterization of Chitosan-Methacrylates (Chi-MAs)

Different chitosan-methacrylates (Chi-MA) as examples of modified chitosan cross-linkers according to the invention were prepared and characterized according to Diolosà et al (Biomacromolecules, 2014, 15, 4606). In brief, 1 g of chitosan (15 kDa and a deacetylation degree of ˜90%) were dissolved in 400 mL of MES 0.05M (pH 5.5). After dissolution, a variable amount of methacrylic acid was added followed by the addition of NHS and EDC ([EDC]/[MAA]=1.5 and [NHS]/[EDC]=1). The solution was stirred for 24 h and the reaction mixture was transferred to dialysis tubes and dialyzed against NaHCO₃ (0.05 M, 3 shifts), aqueous HCl (0.001 M, 2 shifts), aqueous NaCl (0.1 M, 4shifts), and deionized water until the conductivity was below 4 μS at 4° C., and finally freeze-dried before use.

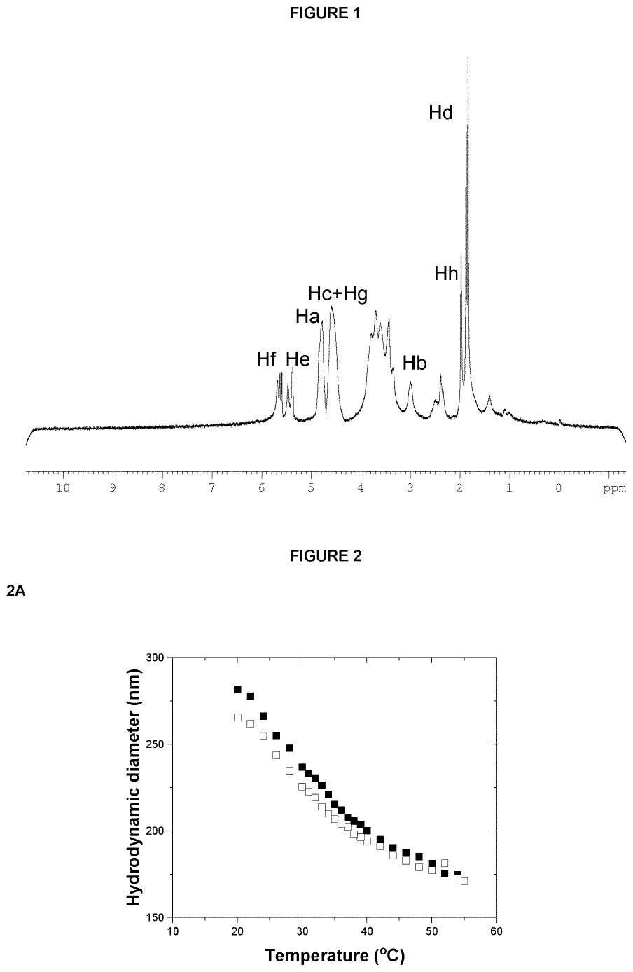

The degree of substitution (DS, i.e., the number of methacrylate groups per chitosan chain on the obtained modified chitosan cross-linkers) was determined by proton nuclear magnetic resonance spectroscopy (1H NMR) in D2O (see FIG. 1). For that, the areas of the signals arising from the vinyl protons of the inserted moieties (at around 5.7 and 55 ppm, respectively) were compared with the signal arising from the anomeric protons of the polysaccharide chain (from 4.5 to 5.1 ppm). In this work, three Chi-MAs were prepared with a DS of 2, 9 and 20, respectively (i.e., Chi2, Chi9 and Chi20).

### Example 2. Synthesis of Microgels Using Chitosan-Methacrylates (Chi-Mas) as Examples of Modified Chitosan Cross-Linkers

Microgels were synthesized by precipitation polymerization in a 250 mL 3-neck round-bottom flask by following the procedure and recipe described by Boularas et al. (Macromolecular Rapid Communications 2015, 36, 79; and Polymer Chemistry 2016, 7, 350) with some modifications. Calculated amounts of different chitosan-methacrylates (Chi-MA) were dissolved in 57.5 mL of water through overnight stirring. Then, MEO₂MA (0.966 g, 5.14 mmol) and OEGMA (0.272 g, 0.573 mmol) were added and the reaction mixture was purged with nitrogen for 45 minutes at room temperature under stirring (150 rpm). The mixture was heated up to 70° C. prior to introduce the KPS solution (14.3 mg dissolved in 2.5 mL of degassed water) into the reactor in order to initiate the polymerization. The reaction mixture became turbid in few minutes and was kept at 70° C. for 6 hours (150 rpm) to complete the reaction. At the end of synthesis, microgels were purified by 3 centrifugation cycles (20,000 rpm, 20 min).

The nomenclature used for the microgels synthesized is based on the type and the molar ratio used of Chi-MAs. In this way, the number following GA-Chi indicates the degree of substitution of chitosan chain, and the value following/indicates the mol % of Chi-MA used with respect to monomers.

TABLE 2
Recipes used for the production of microgels using
Chi-MAs as modified chitosan cross-linkers.

| | [MEO₂MA]₀ | [OEGMA]₀ | [Chi-MA]₀ | [KPS]₀ |
|---|---|---|---|---|
| Microgel | mmol · L⁻¹ | mmol · L⁻¹ | mmol · L⁻¹ | mmol · L⁻¹ |
| GA-Chi20/1* | 83.9 | 9.36 | 0.10 | 0.86 |
| GA-Chi20/0.4 | 83.9 | 9.36 | 0.04 | 0.86 |
| GA-Chi20/0.2 | 83.9 | 9.36 | 0.02 | 0.86 |
| GA-Chi9/2.2* | 83.9 | 9.36 | 0.22 | 0.86 |
| GA-Chi9/1 | 83.9 | 9.36 | 0.1 | 0.86 |
| GA-Chi9/0.4 | 83.9 | 9.36 | 0.04 | 0.86 |
| GA-Chi9/0.2 | 83.9 | 9.36 | 0.02 | 0.86 |
| GA-Chi2/10* | 83.9 | 9.36 | 1 | 0.86 |

Reaction conditions: rpm = 150, reaction temperature = 70° C., reaction time = 6 h.
*Same amount of reactive groups able for cross-linking.

### 2.1. Colloidal Characterization of the Synthesized Microgels

Colloidal characteristics of the microgels synthesized, such as the average hydrodynamic particle diameters at different temperatures and pHs, were measured by Dynamic Light Scattering (DLS) on Zetasizer Nano (Malvern) using 1 mg/mL sample concentration, in all the cases. In all the measurements, the pH was controlled using different buffered media at an ionic strength of 1 mM. For pH-sensitivity, measurements were carried out at 25° C. from pH 3 to 8 and using 5 min as stabilization time. Thermal behavior was studied carrying out measurements every 2° C. from 20 to 55° C., except from 30 to 40° C. that they were carried out per grade. The pH was fixed to 6 for temperature ramps and the stabilization time was 10 min at each temperature.

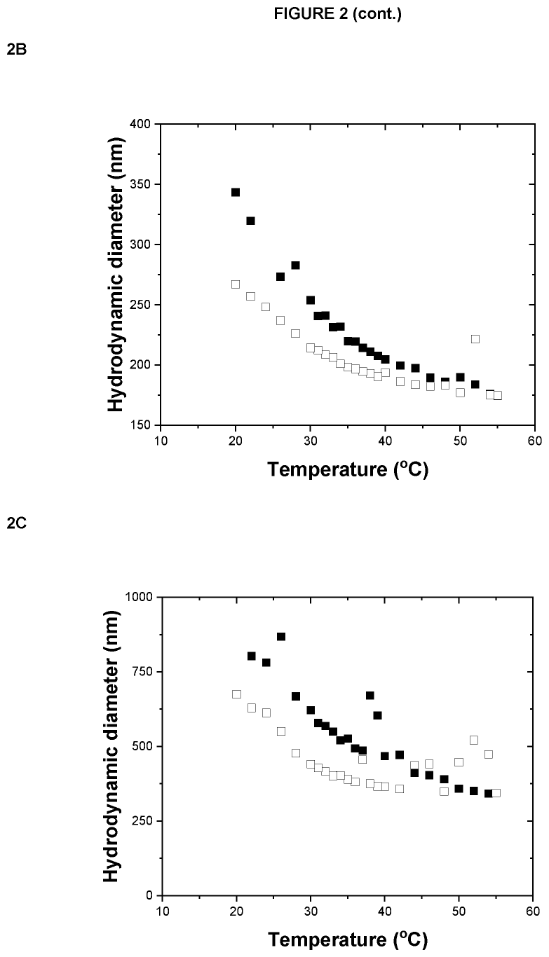

FIG. 2 shows the effect of the Chi-MA20 cross-linker concentration on the thermal behavior of the final microgel particles. The Chi-Ma cross-linker concentration was adjusted to 1 mol % (GA-Chi20/1; 2A), 0.4 mol % (GA-Chi20/0,4; 2B) and 0.2 mol % (GA-Chi20/0.2; 2C) of the total number of MEO₂MA and OEGMA monomers. As shown in FIG. 2, microgel particles swell temperature decreases and shrink at temperatures above the VPTT when increasing the temperature until reaching the collapsed state (Volume Phase Transition Temperature). The VPTT values found were:

$$GA - Chi 20 / 1 microgel - 26.7 ° C .$$

$$GA - Chi 9 / 2.2 microgel - 25.4 ° C .$$

$$GA - Chi 2 / 10 microgel - ∼ 31 ° C .$$

In addition, and mainly below the VPTT, hysteresis is observed between heating and cooling cycles and the differences between both cycles decrease as Chi-MA20 cross-linker concentration increases. This behavior can be explained due to the presence of non cross-linked P (MEO₂MA-OEGMA) copolymer chains.

Even though non-cross-linked P (MEO₂MA-OEGMA) copolymer chains may also be part of the microgel particles, increasing the amount of modified chitosan cross-linker to obtain the microgel particles, makes the copolymer chains more entangled and therefore, the copolymer chains movement is restricted and the hysteresis between cooling and heating cycles decreases.

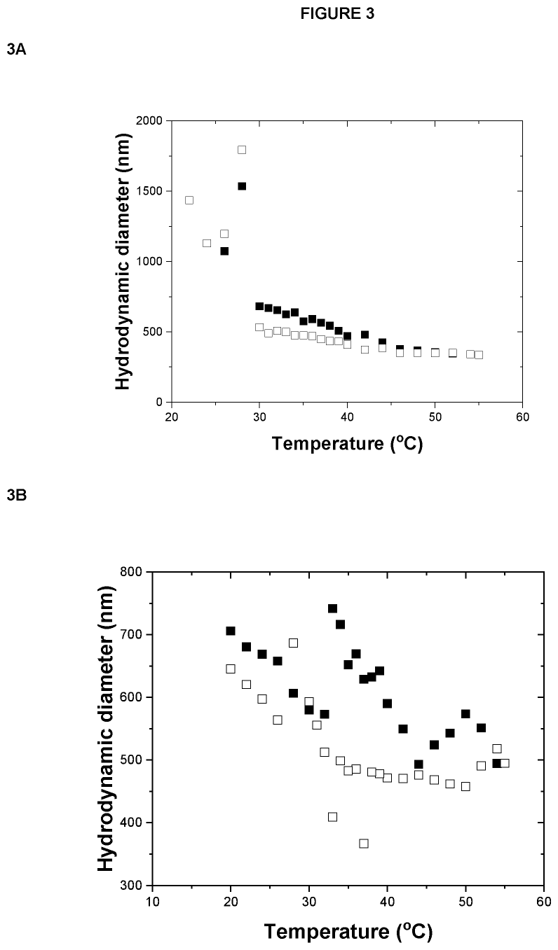

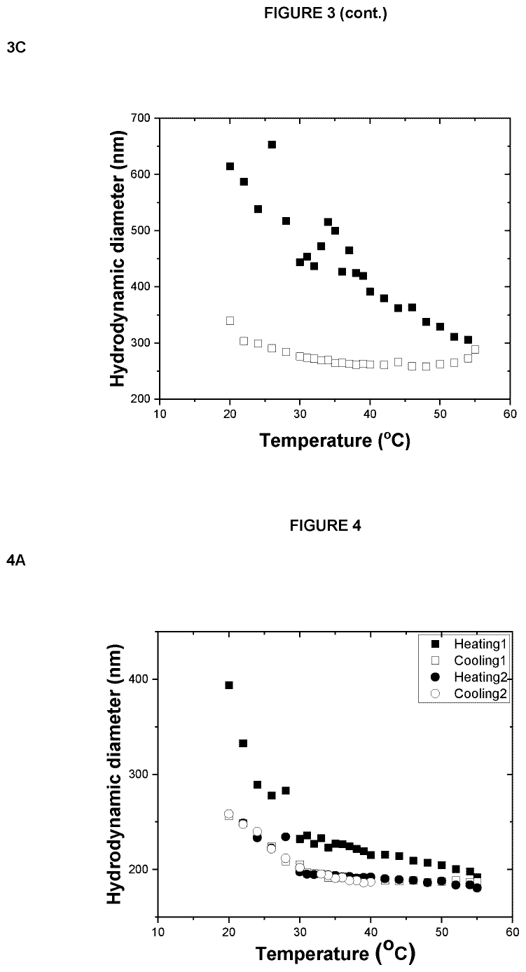

The effect of the DS of the chitosan-methacrylates on temperature sensitivity was analyzed using Chi-MA9 as cross-linker, adjusting the Chi-Ma cross-linker concentration to 1 mol % (GA-Chi9/1), 0.4 mol % (GA-Chi9/0,4) and 0.2 mol % (GA-Chi9/0,2) of the total number of MEO₂MA and OEGMA monomers. It should be noted that the effectiveness of Chi-MA9 as a cross-linking agent was expected to be lower than that of Chi-MA20 because the former had less methacrylate groups per chain able to form cross-linking points. The effect of using Chi-MA9 as a cross-linker on temperature sensitivity of the final microgel particles is illustrated in FIG. 3 showing that all final microgel particles were swollen when decreasing the temperature of the medium, whereas the particles collapsed at temperatures above VPTT, as expected. In addition, as also shown in FIG. 3, microgels synthesized using Chi-MA9 as a cross-linker, also presented hysteresis between heating and cooling cycles.

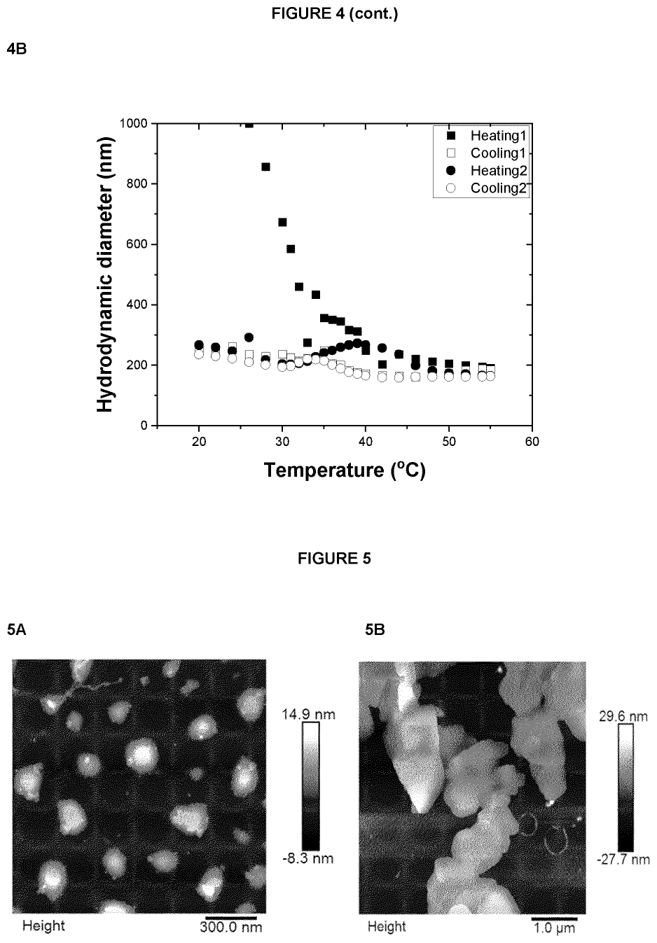

To further analyze the effect of the DS of Chi-MAs in the microgel particle's behavior, a microgel with 10 mol % of Chi-MA2 (GA-Chi2/10) was also synthesized. As shown in FIG. 4, which compares the average hydrodynamic diameters as a function of temperature for GA-Chi9/2.2 and GA-Chi2/10, hysteresis appears between heating-cooling cycles. In addition, hysteresis is higher when decreasing the DS of Chi-MA from 9 to 2 (the GA-Chi9/2.2 microgel was synthesized with 2.2 mol % of Chi-MA9 cross-linker and the microgel GA-Chi2/10 was synthesized with 10 mol % of Chi-MA2cross-linker). In the case of the GA-Chi9/2.2 microgel, the hysteresis between both cycles disappears after the second heating-cooling cycle. It appears that, after having been subjected to a first heating, a reorganization of the copolymer chains occurs which subsequently prevents their movement and accordingly, also prevents the hysteresis between cycles.

In addition, aggregate formation is observed around VPTT for the microgel GA-Chi2/10. This could be related to the distribution of the crosslinking points inside microgel particles. In this case, each chitosan chain only features two methacrylate groups, which could result in higher mobility of said chains than when the chitosan features a higher degree of substitution with methacrylate groups.

In order to confirm the reversible aggregates formation around VPTT, microgel particles at room temperature and at 37° C. were observed by AFM microscopy. Below VPTT well separated and collapsed particles were observed (FIG. 5A). By contrast, around VPTT, some small aggregates were observed confirming the results obtained by DLS (FIG. 5B).

### 2.2. Microstructure Characterization of the Synthesized Microgels by 1H NMR

The microstructure of synthesized microgels GA-Chi20/1, GA-Chi9/2.2 and GA-Chi2/10 was analyzed by high-resolution transverse relaxation (T₂) NMR measurements using the protocol presented by other authors with some modifications (A. Pikabea, et al., Journal of Polymer Science Part A: Polymer Chemistry 2015, 53, 2017; and E. Dieuzy et al., Journal of Colloid and Interface Science 2021, 581, 806). For these measurements 2 wt % of microgels dissolved in deuterated water was used. The temperature was controlled by a Bruker temperature controller, which kept the sample temperature stable at 25° C.

To investigate the microstructure heterogeneity of the different microgel particles, the resonance peak appearing at 3.6 ppm of methylene protons of (PMeO₂MA-OEGMA) chains was used due to its high intensity. Biexponential decay for the integral of methylene protons for the different microgels was assumed. Then, using the equations suggested by Balaceanu et al. (Macromolecules 2011, 44, 2161), short (T_{2S}) and long (T_{2L}) transverse relaxation times together with the relative amounts of methylene protons of (PMeO₂MA-OEGMA) chains in core (C_{S}) and shell (C_{L}) were obtained describing quantitatively the bimodal heterogeneity of the polymer network in microgel particles. In addition, the ratio of the crosslinking densities (CLD^{core}/CLD^{shell}) of the microgels was obtained from the following equation (1):

$$\frac{{CLD}^{core}}{{CLD}^{shell}} = {( \frac{{T}_{2 L}}{{T}_{2 S}} )}^{1 / 2} \qquad ( 1 )$$

In addition, applying the extended Flory-Rehner theory to analyze the inner structure of stimuli-responsive microgels,⁵ it is possible to obtain the number of chains between two crosslinking points, i.e. subchains, from the following relationship (2):

$$\frac{{N}^{core}}{{N}^{shell}} = \frac{{C}_{S}}{{C}_{L}} {( \frac{{T}_{2 L}}{{T}_{2 S}} )}^{1 / 2} \qquad ( 2 )$$

Moreover, the number of subchains in the microgel particles can be estimated considering that each modified chitosan cross-linker molecule connects two subchains with the equation (3):

$$N = 2 {N}_{A} {v}_{0} c \qquad ( 3 )$$

where N_{A} is the Avogadro's constant, v₀ is the volume of each microgel particle and c is the molar concentration of cross-linker. Using the data obtained from above formulas, it is possible to calculate the proportionality parameter (p) between the radius of the core and the hydrodynamic radius of the microgel particles from the equation (4):

$$N = \frac{1}{2} ( \frac{{N}^{core}}{{ρ}^{3}} + \frac{{N}^{shell}}{1 - {ρ}^{3}} ) \qquad ( 4 )$$

The relative amounts of methylene protons of (PMeO₂MA-OEGMA) (C_{S} and C_{L}), the ratios of crosslinking densities (CLD^{core}/CLD^{shell}), the number of subchains in the core (N^{core}), number of subchains in the shell (N^{shell}) and the ratio between the radius of the core and the hydrodynamic radius of the microgel particles (p) are reported in Table 3. As can be observed, in all the cases core-shell microstructure is confirmed being the number of subchains in the shell lower except in the case of the microgel synthesized with slightly substituted chitosan. The reason for the higher amount of subchains in the core than in the shell is due to the higher amount of cross-linking points in the core in the case of using highly substituted chitosans as cross-linker. By contrast, when slightly substituted cross-linker is used, the cross-linking points distribution changes completely being more cross-linked the shell than the core. Finally, regarding the volume of the core, in the case of microgels synthesized with highly substituted chitosans, similar value than for oligo (ethylene glycol)-based microgel synthesized with conventional cross-linkers was obtained. Moreover, decreasing the substitution degree of chitosan, the volume of the core of particles decreases. In summary, the possibility of tuning final microgel particles' microstructure as a function of Chi-MA type has been corroborated.

TABLE 3
Relative weight coefficient of (PMeO₂MA-OEGMA) chains in the core
(C_{S}) and shell (C_{L}), crosslinking density ratios (CLD^{core}/CLD^{shell}), number
of subchains in the core (N^{core}) and in the shell (N^{shell}) and
volume of the core (%) as a function of Chi-MA type.

| | | | | | | Volume |
|---|---|---|---|---|---|---|
| | | | CLD^{core}/ | | | of the |
| Cross-linker | C_{S} | C_{L} | CLD^{shell} | N^{shell} | N^{core} | core (%) |
| Chi-MA20 | 0.41 | 0.59 | 4.1 | 168474 | 480151 | 62 |
| Chi-MA9 | 0.35 | 0.65 | 4.0 | 261044 | 561244 | 59 |
| Chi-MA2 | 0.18 | 0.82 | 3.8 | 303443 | 251858 | 48 |

### 2.3. Enzymatic Degradation of the Synthesized Microgels

To evaluate the biodegradability of the microgels enzymatic degradation tests with lysozyme were carried out.

Enzymatic degradation of microgel particles synthesized in the previous examples (GA-Chi20/1, GA-Chi9/2 and GA-Chi2/10) by lysozyme was carried out following the conditions reported by Diolosà et al. (Biomacromolecules, 2014, 15, 4606) with some modifications. Microgel particles were lyophilized and resuspended in PBS (pH 7,4, 10 mM) buffer at 1 mg/mL concentration. Then, lysozyme was added at 1:38 ratio (w/w) and degradation was studied at 37° C. and pH 7.4 in order to mimic physiological conditions. At different incubation times, a sample was taken and the size of microgel particles was analyzed directly by DLS. After, the same samples were filtered and the presence of possible oligosaccharides was determined by DLS.

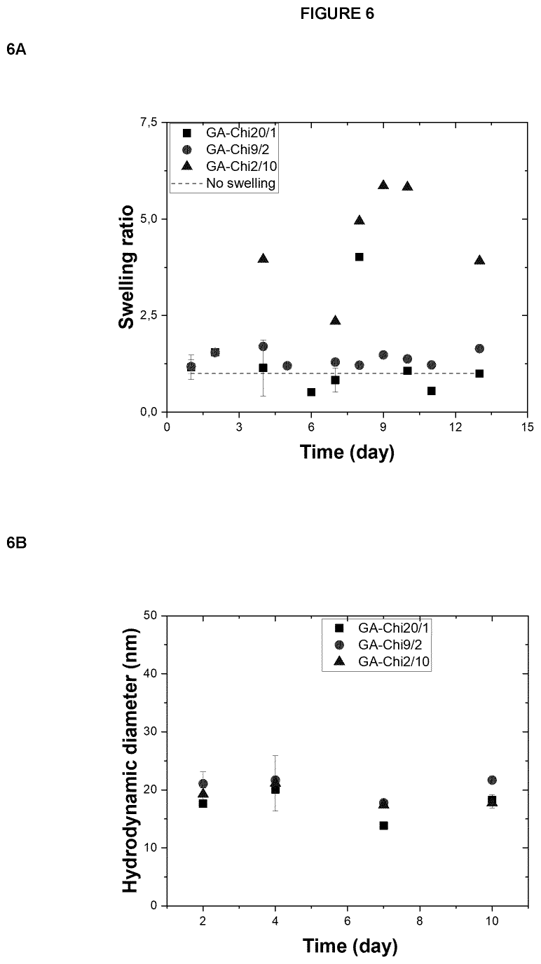

The swelling ratio of the different microgels (GA-Chi20/1, GA-Chi9/2 and GA-Chi2/10) and the presence of oligosaccharides at different incubation times was studied. As shown in FIG. 6, swelling of microgel particles is observed only for the synthesized microgels having Chi-MA cross-linkers with a low degree of methacrylate substitution, meaning that the enzyme is able to cleave the chitosan chains, increasing therefore the swelling ability of the microgel particles (FIG. 6A). In addition, the presence of oligosaccharides is detected for all the microgels (FIG. 6B). It is known that the presence of methacrylate units reduces the rate of chitosan chain cleavage since their presence impairs the recognition in the enzyme cleft. Accordingly, the reason behind the swelling of the microgels synthesized featuring slightly substituted Chi-MA could be the higher release of oligosaccharides than when the microgels are synthesized with highly substituted Chi-MAs.

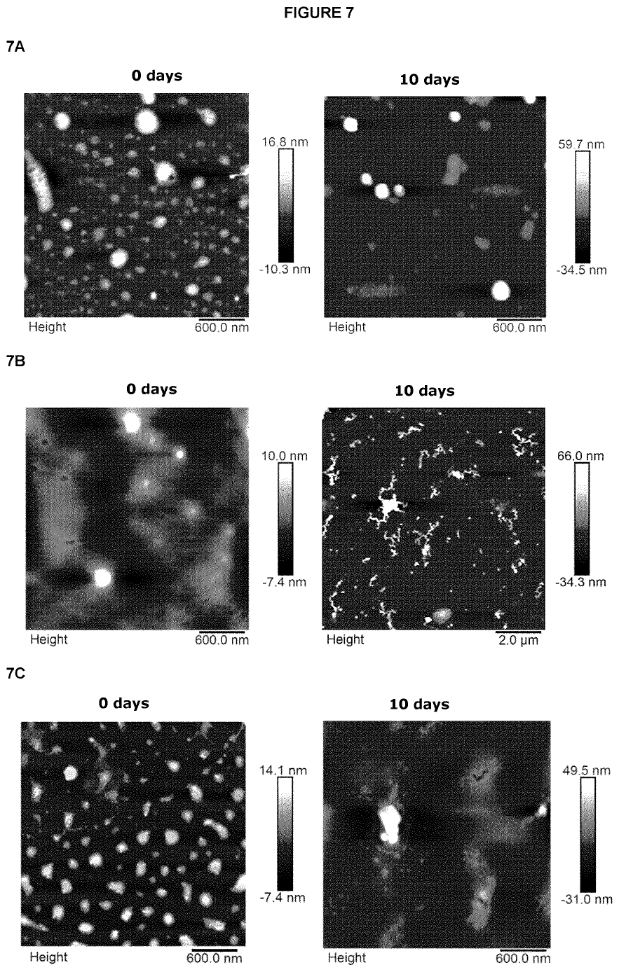

With the aim of understanding better the degradation of microgel particles, the morphology of different synthesized microgels (GA-Chi20/1, GA-Chi9/2 and GA-Chi2/10) was analyzed by AFM at different incubation times. In that connection, microgel particles were incubated with lysozyme and samples were taken at time 0 and after 10 days of incubation. As observed in FIG. 7, at the beginning of the incubation spherical particles are observed in all the cases. By contrast, after 10 days of incubation, microgel particles have lost their particle identity for microgels crosslinked with Chi-MAs having low DS (Chi-MA2 and Chi-MA9), corroborating their degradation and the release of oligosaccharides.

### 2.4. Encapsulation of Active Molecules by the Synthesized Microgels

The loaded amounts of benzophenone-4 into microgel particles of the invention (GA-Chi9/2,2 and GA-Chi2/10) were determined using the “hydrophobic” method described previously (G. Aguirre et al., Polymer Chemistry 2018, 9, 757; and Colloids and Surfaces B: Biointerfaces 2019, 175, 445). Briefly, microgel dispersions (1 mg/ML) with different pHs (pH 4 and 6) were heated to and incubated at 50° C. (above the volume phase transition temperature, VPTT) for 30 min. To this microgel dispersion benzophenone-4 in ethanol (1 mg/mg_{microgel}) and preheated were added under magnetic stirring. After that, the mixed dispersion was stirred overnight at 50° C. to remove the organic solvent. After separating microgel particles from the aqueous medium containing free active molecules through centrifugation, the equilibrium benzophenone-4 concentration was determined by UV-Vis.

Encapsulation efficiency (E.E.) was calculated as follows:

$$E . E . \% = \frac{weight of active molecule in microgel dispersion}{weight of feeding active molecule} \times 100 \qquad ( 5 )$$

As can be seen in Table 4, lower encapsulation efficiencies than in the case of classical microgels are obtained, in all the cases. Moreover, it seems that nor the microstructure of microgel particles neither the pH of the medium have effect on Benzophenone-4 encapsulation. This is an unexpected result since at pH 4 microgel particles are swollen and positively charged and being Benzophenone-4 molecules negatively charged, encapsulation by electrostatic attraction forces should be enhanced. In addition, Benzophenone-4 has an aromatic ring in its structure and therefore, hydrophobic interactions would be expected between them and ethylene/methylene groups of microgel particles. However, it seems that electrostatic attraction forces and hydrophobic interactions are not enough to encapsulate Benzophenone-4 molecules efficiently into microgel particles using this protocol.

The hysteresis shown by the microgels may have an effect on the release of the encapsulated molecules since the colloidal state of microgel particles will be different depending on the heating or cooling cycle. However, as previously shown, this hysteresis can be controlled and avoided using acceptable cross-linker concentrations.

TABLE 4
Encapsulation efficiencies (E.E.) as a function
of Benzophenone-4 for microgel particles.

E.E. (%)

| Microgel | pH 4 | pH 6 |
|---|---|---|
| GA-Chi9/2.2 | 20.4 ± 6.5 | 18.6 ± 6.4 |
| GA-Chi2/10 | 12.4 ± 5.0 | 18.6 ± 4.9 |

### Example 3. Microgels Self-Assembled Films

### 3.1. Films Formation

Self-assembled microgel films GA-Chi20/1, GA-Chi9/2.2 and GA-Chi2/10 were formed via an easy handling procedure based on water evaporation (G. Aguirre et al., Polymer Chemistry 2018, 9, 1155). Briefly, non-purified microgel dispersion was placed in a silicon mold and dried at 35° C. (+3° C.) and atmospheric pressure in a bell jar oven. A non-transparent film is obtained may be due to the presence of precipitated Chi-MA free chains (WSP). Nevertheless, after immersion in aqueous solution, the film is able to swell and maintains its film identity confirming the formation of a cohesive film.

With the aim of improving the transparency of the films, free Chi-MA chains were removed by centrifugation or dialysis. After two purification methods, the films obtained are transparent.

Therefore, it seems that the purification step is necessary to obtain conventional thermal behavior and transparent films.

This means that microgel particles of the present invention, featuring a DS of at least 2 and at least 0.2 mol % of Chi-MA cross-linker, show non-conventional thermal behavior, i.e., the microgel particles collapse progressively within a range of temperatures, which may allow the use of the microgels of the invention as slow-release delivery systems.

### 3.2. Enzymatic Degradation

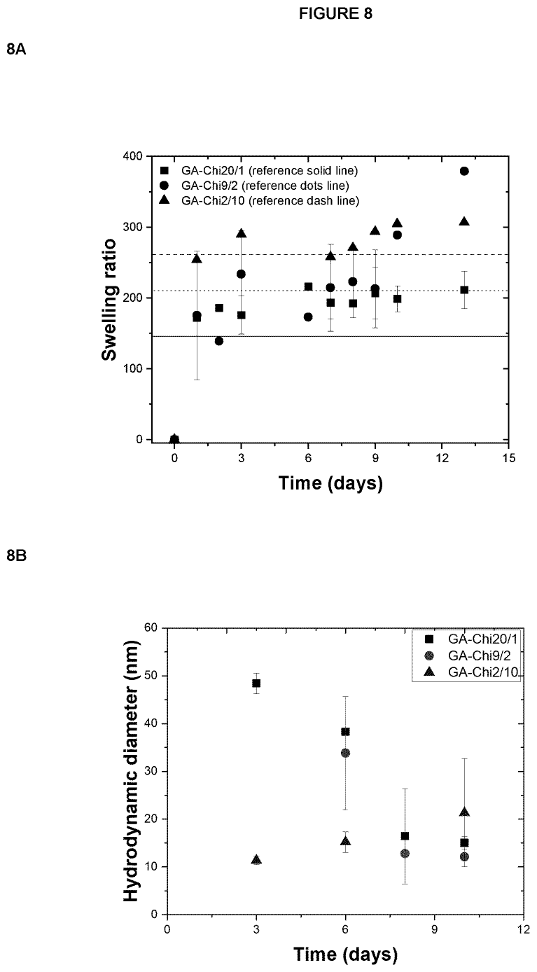

The enzymatic degradation of films by means of lysozyme was carried out following the same conditions used for microgel particles. In this case, it was not necessary to filter the samples before analyzing by DLS to determine the presence of oligosaccharides since the films maintained their identity during whole incubation time. FIG. 8 shows the swelling ratio of films prepared with microgels GA-Chi20/1, Ga-Chi9/2 and Ga-Chi2/10 and the presence of oligosaccharides at different incubation times. As seen in FIG. 8, after the incubation with enzyme, swelling of the film is observed, in all the cases. This means that the enzyme is able to cleave the Chi-MA chains, increasing the swelling ability of the films. Moreover, it seems that in the case of slightly substituted chitosan, the swelling ratio increases more than in the other cases. The reason could be, as in the case of microgel particles, the higher release of oligosaccharides even if oligosaccharides have been detected for all the films (FIG. 8B).

### 3.3. Encapsulation of Active Molecules

The encapsulation of Benzophenone-4 into self-assembled microgel films was carried out following the protocol described previously with some modifications (G. Aguirre et al., Polymer Chemistry 2018, 9, 1155). The loaded amounts of the active molecule into films were determined immersing the films in Benzophenone-4 buffered aqueous solutions (1 mg/mg_{film}) with different pHs (4 and 6) and allowing them to rehydrate during 24 h at room temperature. Then, the loaded-films were separated and the solutions containing non-encapsulated Benzophenone-4 molecules were analyzed by UV-Vis to determine the amount of non-encapsulated active molecules. In Table 5, encapsulation efficiency (E.E.) values obtained as a function of pH are presented. As can be observed, higher E.E. than in the case of isolated particles are obtained but lower than that observed for classical films,¹⁰ in all the cases. The reason for this could be that the Benzophenone-4 is adsorbed on the surface of the films and not encapsulated into them. Moreover, in the case of films formed with microgels with conventional core-shell microstructure (GA-Chi20/1 and GA-Chi9/2.2), decreasing the pH of the medium the E.E. decreases. It seems that electrostatic attraction forces are not enough to gain to the decreasing of hydrophobic interactions, i.e., at pH 4 films are more hydrophilic. By contrast, in the case of the films formed with microgels with inverse core-shell microstructure (GA-Chi2/10), decreasing the medium pH, E.E. increases. In the case of this microgel particles, Chi-MA chains are mainly located at the surface of the particles and therefore, the electrostatic attraction forces are strong enough to improve the E.E. of Benzophenone-4.

TABLE 5
Encapsulation efficiencies (E.E.) as a function of Benzophenone-4
for self-assembled microgel particles.

E.E. (%)

| Film | pH 4 | pH 6 |
|---|---|---|
| GA-Chi20/1 | 34.0 ± 1.8 | 45.5 ± 1.1 |
| GA-Chi9/2.2 | 32.8 ± 0.8 | 46.0 ± 0.4 |
| GA-Chi2/10 | 67.5 ± 1.1 | 49.8 ± 2.1 |

In order to study the ability of films to encapsulate a broad spectrum of molecules, the encapsulation of macromolecular hyaluronic acid into them was studied using the same encapsulation protocol presented above. To determine the free hyaluronic acid molecules, IR technique was used but unfortunately, the resolution of the spectra was not good enough to obtain accurate results. Therefore, ¹H NMR was used to determine non-loaded macromolecules.

As seen in Table 6, films are able to encapsulate hyaluronic acid but as a function of the microstructure different conditions are needed. In the case of the films formed with microgels with conventional core-shell microstructure (GA-Chi20/1 and GA-Chi9/2.2) hydrophobic interactions are the main driving force for the encapsulation. By contrast, in the case of the films formed with the microgels with the inverse core-shell microstructure, electrostatic attraction forces are predominant during the encapsulation. In summary, self-assembled microgel films are able to encapsulate small active molecules and also macromolecules.

TABLE 6
Encapsulation efficiencies (E.E.) as a function of hyaluronic
acid for self-assembled microgel particles.

E.E. (%)

| Film | pH 4 | pH 6 |
|---|---|---|
| GA-Chi20/1 | 0 | 49.3 ± 11.1 |
| GA-Chi9/2.2 | 0 | 44.5 ± 9.7 |
| GA-Chi2/10 | 32.4 ± 4.2 | 0 |

### 3.4. Mechano-Electrical Properties

Mechano-electrical properties of films made with GA-Chi20/1 and GA-Chi9/2.2 microgels were measured using the following method. An Indium Tin Oxide (ITO) slide covered with adhesive tape, saving a small part for film deposition, was deposited onto a force sensor. Then, copper tape was fixed to a finger in order to use it as a second electrode. Finally, the films were compressed smoothly with the finger during few seconds and the output voltage and the force applied was recorded by using LabVIEW software. Output voltages higher than 250 mV were generated by compression and maintained constant almost for 1 minute.

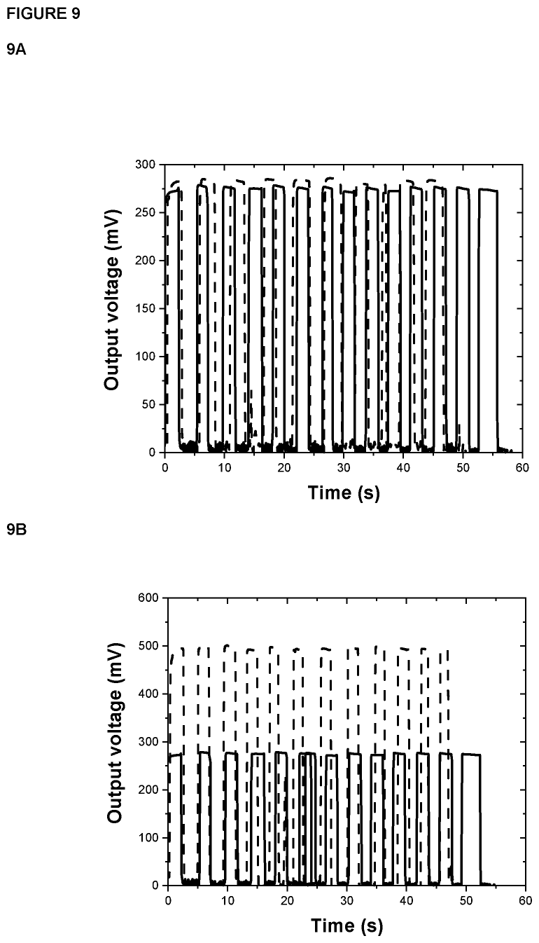

In addition, with the aim of increasing the electrical potential generated, two film units were connected in series. The applied force was controlled and fixed to 10 to 15 N, being similar to a finger pressure for cream application. In FIG. 9, the output voltage generated for 1 film and 2 films connected in series are shown. As can be seen, for the film formed with GA-Chi9/2.2 microgel particles the output voltage generated is higher, being this difference more important in the case of films connected in series. The reason is the higher swelling ability of the GA-Chi9/2.2 film (210%) than GA-Chi20/1 film (146%). Increasing the swelling ability of the film, the mobility of the ions is enhanced by increasing the output voltage generated. In addition, in the case of the films connected in series, a high electrical potential of 0.5 V can be generated (FIG. 9B) making it possible to amplify and linearly tune it combining the appropriate number of films in series.

### 3.5. Mechanical and Adhesive Properties

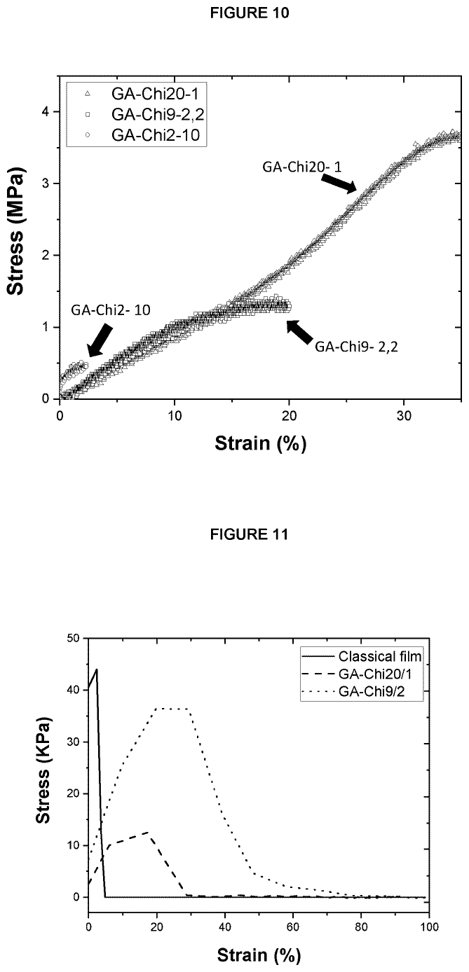

Tensile tests were performed on a texture analyzer TA.XT Plus from Stable Micro Systems at a speed of 1 mm·s⁻¹ for films formed with microgels of the invention GA-Chi20/1, GA-Chi9/2,2 and GA-Chi2/10. Film dimensions were in the following range: 150-200 μm×10 mm×25 mm. Young's modulus was calculated from the elastic region derived from the first linear slope of the stress-strain curve which here corresponded to strain from 0 and 8%, except for GA-Chi2/10 film. The stress versus strain curves for films are reported in FIG. 10. Young's modulus, the tensile strength (equivalent to the tensile stress at break in this case), the strain at break and the fracture energy were extrapolated from the curves (Table 7).

Young's moduli and tensile strengths films are lower of these compared to the ones reported for classical films cross-linked with oligo (ethylene glycol) diacrylate (OEGDA) and N—N′-methylenebisacrylamide (MBA) but appropriate for skincare applications. In addition, low elongation at break is observed and it decreases with Chi-MA concentration, i.e., decrease of the DS of Chi-MA together with the fracture energy. Regarding the elongation at break values obtained, a strong impact due to the type of Chi-MA used as cross-linker was observed. Decreasing the DS of Chi-MA from 20 to 2, the elongation at break value decreased dramatically from 35 to 2%. The reason behind this effect is related to the microstructure of the microgels. The inverse core-shell microstructure (highly cross-liked shell and slightly cross-linked core) of microgels cross-linked with Chi-MA2 lead to tighter films.

TABLE 7
Strain at break, stress at break and
fracture energy of different films.

| | GA-Chi20-1 | GA-Chi9-2.2 | GA-Chi2-10 |
|---|---|---|---|
| Young's modulus | 0.11 ± 0.03 | 0.13 ± 0.03 | 0.23 ± 0.11 |
| (MPa) | | | |
| Tensile strength | 3.3 ± 0.4 | 1.6 ± 0.4 | 0.4 ± 0.1 |
| (MPa) | | | |
| Strain at break (%) | 35 ± 3 | 22 ± 2 | 2.5 ± 0.7 |
| Fracture energy | 60 ± 9 | 19 ± 3 | 0.8 ± 0.1 |
| (J · m⁻³) | | | |

Regarding the adhesive properties of the different films, films formed with microgels of the invention GA-Chi20/1 and GA-Chi9/2 were analyzed using probe-tack method. For that, a flat ended probe came in contact with the film placed on a glass substrate. After contact in controlled pressure (10 N) during 60 s, the probe was pulled back from the film (500 μm/s). During the test, the forced required to separate the probe and its displacement were recorded. From this data, the stress/strain curve were plotted (FIG. 11). As can be observed, using Chi-MA as cross-linker, the adhesive properties of the films are better than that observed for classical film. In addition, decreasing the DS of the Chi-MA and hence, increasing its concentration into microgel particles, films present better adhesive properties and adhesive failure. In the case of the films formed with microgels with inverse core-shell microstructure, no adhesive properties are observed. Therefore, regarding the mechanical and adhesive properties of the films, it has been observed that the microgels' microstructure has a strong impact on them.

1. A microgel composition comprising microgel particles, wherein said microgel particles comprise oligo (ethylene glycol)-based copolymers crosslinked with a modified chitosan cross-linker obtainable by reaction of a chitosan with monomers comprising —COOH or —COO⁻M⁺ groups, M⁺ representing a cation.

2. The microgel composition according to claim 1, wherein the modified chitosan cross-linker comprises substitutions selected from the group consisting of acrylamide, methacrylamide, acrylate, and methacrylate groups, or a mixture thereof.

3. The microgel composition according to claim 1, wherein said modified chitosan cross-linker is a chitosan comprising at least 2 substitutions, per each chitosan polymer chain, being selected from the group consisting of acrylamide, methacrylamide, acrylate groups, and methacrylate groups, or a mixture thereof.

4. The microgel composition according to claim 1, wherein the oligo (ethylene glycol)-based copolymers comprise di(ethylene glycol) methyl ether groups and oligo (ethylene glycol) methyl ether groups having from 3 to 12 ethylene glycol units.

5. The microgel composition according to claim 1 produced by aqueous phase-precipitation polymerization of one or more of the following monomers:
di(ethylene glycol) methyl ether methacrylate (MeO₂MA);
an oligo(ethylene glycol) methyl ether methacrylate (M(EO)ₙMA), n being an integer ranging from 3 to 12;
cross-linked with a modified chitosan cross-linker by reacting a chitosan with one or more monomers of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation.

6. The microgel composition according to claim 5 produced by via aqueous phase precipitation polymerization of monomers cross-linked with a modified chitosan cross-linker, said monomers being di(ethylene glycol) methyl ether methacrylate (MeO₂MA), oligo(ethylene glycol) methyl ether methacrylate (OEGMA); and wherein the modified chitosan cross-linker is obtained by reacting a chitosan with methacrylic acid (MAA) monomers.

7. The microgel composition according to claim 5, wherein the molar % of the modified chitosan cross-linker is between 0.1% to 20% with respect to the total number of moles of monomers.

8. A process of preparing a microgel composition according to claim 1, said process comprising preparing a microgel via precipitation polymerization of monomers selected among:
di(ethylene glycol) methyl ether methacrylate (MeO₂MA);
an oligo (ethylene glycol) methyl ether methacrylate (M(EO)ₙMA), n being an integer ranging from 3 to 12;
wherein said monomers are crosslinked with a modified chitosan cross-linker obtainable by reacting a chitosan with one or more monomers of formula CR₁R₂═CR₃R₄ in which R₁, R₂, R₃ and R₄ represent a hydrogen, a halogen or a hydrocarbon group, at least one of the four groups comprising a —COOH or —COO-M⁺ group, M⁺ representing a cation;
and wherein said microgel comprises particles.

9. A Self-Assembled Microgel Film produced by solvent evaporation of a microgel composition according to claim 1.

10. A Self-Assembled Microgel Film produced by applying a microgel composition according to claim 1 on keratin materials.

11. A cosmetic product comprising a microgel composition according to claim 1 and at least a cosmetic agent, wherein the particles of the microgel comprise the cosmetic agent.

12. A make-up or a skin care method comprising a step of applying on keratinous materials a cosmetic product according to claim 11 and applying a compression on said cosmetic product.

13. A therapeutic product comprising a microgel composition according to claim 1 and a therapeutic agent, wherein the particles of the microgel comprise the therapeutic agent.

14. A therapeutic method comprising delivering a subject the microgel composition according to claim 1.

15. A series of films obtained by drying or evaporating solvent of a microgel composition according to claim 1 wherein each film is connected to another film.

16. A series of Self-Assembled Microgel Films obtained by drying or evaporating a solvent of a microgel composition in a Self-Assembled Microgel Film according to claim 9 such that each Self-Assembled Microgel Film is connected to another Self-Assembled Microgel Film.

17. The microgel composition of claim 4 wherein the oligo (ethylene glycol) methyl ether groups have 8 to 10 ethylene glycol units.

18. The microgel composition of claim 5 wherein n ranges from 8 to 10.

19. The process of claim 8 wherein n ranges from 8 to 10.
